(19) United States
(12) Patent Application Publication
(10) Pub. No.: US 2026/0328474 A1
(43) Pub. Date: Oct. 22, 2026

(54) SPRAY DRYING SYSTEMS AND METHODS

(71) Applicant: Celanese International Corporation, Irving, TX (US)

(72) Inventors: Spencer Henry Bowen, League City, TX (US); Elizabeth A. Poole, League City, TX (US); Rudolphus Dirken, Gunzwil (CH)

(73) Assignee: Celanese International Corporation, Irving, TX (US)

(21) Appl. No.: 19/184,236

(22) Filed: Apr. 21, 2025

(51) Int. Cl. F26B 3/12; A61K 9/16; B08B 5/02

(52) U.S. Cl. CPC F26B 3/12; A61K 9/1682; B08B 5/02

(57) ABSTRACT

Disclosed is a spray drying device, methods for spray drying, and a method for cleaning a particle collection device. The spray drying device and methods utilize a conveying medium and/or cleaning medium for the conveyance of an anti-blocking agent and for cleaning of the particle collection device, respectively. The conveying medium and/or cleaning medium comprises an inert gas in an amount greater than 90 vol. %. The conveying medium and/or cleaning medium advantageously provide increased efficiency and safety for spray drying techniques.

### FIELD OF THE INVENTION

This disclosure relates generally to the field of spray drying. In particular, the present disclosure relates to the use of a conveying medium to convey anti-blocking agent in a rotary spray dryer system, a process for producing a dispersion powder, and a method for cleaning a particle collection device.

### BACKGROUND OF THE INVENTION

Pharmaceutical and chemical industries utilize spray drying to produce particles with controlled particle size and properties. Generally, spray drying devices comprise a drying chamber, a means for dispersing a material, a drying gas, and a means for collecting the particles produced thereby. Spray drying produces finely dispersed particles as well as non-product particles commonly referred to in the art as “dust” or “fines,” i.e., finely dried particles that do not ultimately coalesce during the spray drying process. Dust creates significant issues for spray drying techniques in terms of safety and efficiency.

Conventional spray drying devices use compressed air to convey materials into the device. This conveyance medium can create safety concerns as feedstocks and additives used in spray drying often contain materials that may be combustible. The likelihood of combustion increases as the materials are dispersed into fine particles, which have high surface area. Combustion may result from the generation and subsequent release of a charge build up, resulting from particles rubbing against one another and/or components of the device. The increased surface area exposes more of the material to any oxygen that may be present, resulting in increased potential for combustion and fire. Additionally, multiple conveyance lines may be used to direct materials into the spray drying device, providing pathways for fire to spread if ignition within the device occurs.

In addition to many commonly used spray drying materials being combustible, they may also be hygroscopic, i.e., they absorb and retain water. This property can undesirably lead to agglomeration of particles and/or additives during the spray drying process. As agglomerates form, less additive may be available to contact the dispersed material, and the device may become clogged, thereby decreasing efficiency.

The need therefore exists for spray drying processes that improve safety and reduce agglomeration.

### SUMMARY OF THE INVENTION

Spray drying technology allows for high throughput production of free-flowing powders with controlled particle size and properties. Additives may enhance the efficiency of the spray drying process and may also improve the properties of the particles produced therefrom. Conventional spray drying techniques use compressed air to convey additives, a method which may ultimately reduce spray drying efficiency and result in safety concerns. For example, the moisture content of compressed air may result in additive agglomeration, decreasing both spray drying efficiency and product particle quality. As another example, many additives are combustible, a property that is heightened when dispersed, i.e., surface area increases. The oxygen content of compressed air further compounds combustibility, increasing the likelihood and severity of a possible explosion.

In some embodiments, the present disclosure relates to a rotary spray dryer system, comprising a vessel defining a drying chamber, wherein the vessel comprises a rotary atomizer configured to atomize an aqueous polymer dispersion to form an atomized dispersion; a drying medium inlet in fluid communication with the drying chamber configured to dry the atomized dispersion with a drying medium and form particles; and an anti-blocking agent inlet configured to deliver an anti-blocking agent to the drying chamber with a conveying medium comprising an inert gas in an amount greater than 90 vol. %, based on the total volume of the conveying medium. In some embodiments, the anti-blocking agent inlet may be oriented adjacent to the rotary atomizer. The anti-blocking agent inlet optionally comprises a plurality of separate conduits. Typically, the rotary spray drying system further comprises a particle collection device comprising a filter housing and a bag filter.

In some embodiments, the present disclosure describes a process for producing a dispersion powder. The process for producing a dispersion powder may comprise atomizing a quantity of an aqueous polymer dispersion and adding an anti-blocking agent in a rotary spray dryer, wherein the anti-blocking agent may be conveyed by a conveying medium comprising an inert gas in an amount greater than 90 vol. %, based on the total volume of the conveying medium. In various embodiments, the anti-blocking agent comprises at least one of functional additives, finished product rework, filler, or a combination thereof.

In some embodiments, the present disclosure relates to a method for cleaning a particle collection device, e.g., a particle collection device in a spray drying system, comprising providing a burst of a cleaning medium to a particle collection device, wherein the cleaning medium comprises an inert gas in an amount greater than 90 vol. %, based on a total volume of the cleaning medium.

In various optional embodiments, the inert gas may comprise a noble gas. For example, the conveying medium may be helium and/or argon. In some aspects, the inert gas may comprise nitrogen. The conveying medium and/or cleaning medium also is preferably dry, meaning it has a water concentration no greater than 80 wppm. In some embodiments, the conveying medium and/or cleaning medium has a water concentration no greater than 80 wppm. In some embodiments, the conveying medium and/or cleaning medium may have a dew point temperature no greater than −40 °C. The conveying medium and/or cleaning medium optionally has an oxygen concentration less than about 1.000 vol. %, e.g., from 0.00001 to 1.000 vol. %, based on the total volume of the conveying medium and/or cleaning medium.

### BRIEF DESCRIPTION OF THE DRAWINGS

The present disclosure will be better understood in view of the appended non-limiting figures, in which:

FIG. 1 provides a cutaway view of a rotary spray dryer system according to some embodiments of the present disclosure;

FIG. 2 provides a cutaway view of a further exemplary rotary spray dryer system according to some embodiments of the present disclosure;

FIG. 3 provides an overview of a process for producing a dispersion powder according to some embodiments of the present disclosure; and

FIG. 4 provides an overview of a method for cleaning a particle collection device according to some embodiments of the present disclosure.

### DETAILED DESCRIPTION OF THE INVENTION

### Introduction

Spray drying is the process of taking a liquid and/or slurry feed solution, typically an emulsion or dispersion, such as an aqueous polymer dispersion, and transforming it into a dried powder. Spray drying comprises atomizing the dispersion into fine droplets within a drying chamber, wherein the droplets are contacted with a drying medium such as air. The contacting causes rapid evaporation of liquid in the dispersion, resulting in the formation of solid product particles. Described herein are novel spray drying devices and methods that preferably exhibit a combination of enhanced efficiency and safety compared to conventional spray drying technology. A method of cleaning a spray drying particle collection device is also described. In some aspects, the spray drying device and methods described herein incorporate a conveying medium and/or cleaning medium comprising an inert gas in an amount greater than 90 vol. % for conveying anti-blocking agents and for cleaning, respectively.

Anti-blocking agents are components that may be added during the spray drying process along with the liquid feed and are provided to reduce agglomeration of the particles as they are formed. That is, mixing of the anti-blocking agent with the feed solution during formation of the product particles may inhibit or prevent the resulting product particles from sticking together during the spray drying process. For example, anti-blocking agents may be used to ensure individual particles remain separated by creating a barrier between individual particles, thereby forming a reliably flowable powder. The inventors have discovered that the use of a conveying medium comprising an inert gas in an amount greater than 90 vol. % for conveying anti-blocking agents ameliorates safety concerns and allows for efficient conveyance of anti-blocking agents during the spray drying process.

Dust, i.e., fine particles, comprising uncoalesced anti-blocking agent and liquid feed, may be generated during spray drying contaminating the product stream. A particle collection device comprising bag filters may be employed to separate the production stream from the conveying medium, ultimately leading to product particle and dust accumulation in the particle collection device. Product particle and/or dust accumulation in the particle collection device undesirably may reduce airflow through the spray drying device. The inventors have also discovered that providing one or more bursts of a cleaning medium comprising an inert gas in an amount greater than 90 vol. % provides an efficient and safe method for cleaning particle collection devices.

### Conveying and Cleaning Mediums

In one aspect, the disclosure relates to a conveying medium comprising an inert gas in an amount greater than 90 vol. %, based on the total volume of the conveying medium. For example, the conveying medium may comprise the inert gas in an amount greater than 90 vol. %, greater than 91 vol. %, greater than 92 vol. %, greater than 93 vol. %, greater than 94 vol. %, greater than 95 vol. %, greater than 96 vol. %, greater than 97 vol. %, greater than 98 vol. %, greater than 99 vol. %, or about 100 vol. %. In terms of ranges, the conveying medium optionally comprises an inert gas in an amount from 90 to 100 vol. %, e.g., from 92 to 100 vol. %, from 95 to 100 vol. % or from 98 to 100 vol. %.

In some embodiments, the disclosure relates to providing a burst of a cleaning medium to a particle collection device to clean the particle collection device. In this aspect, the cleaning medium of the disclosure may comprise the inert gas in any of the aforementioned amounts concerning the conveying medium. That is, the cleaning medium may comprise the inert gas in an amount greater than 90 vol. %, greater than 91 vol. %, greater than 92 vol. %, greater than 93 vol. %, greater than 94 vol. %, greater than 95 vol. %, greater than 96 vol. %, greater than 97 vol. %, greater than 98 vol. %, greater than 99 vol. %, or about 100 vol. %. In terms of ranges, the cleaning medium optionally comprises an inert gas in an amount from 90 to 100 vol. %, e.g., from 92 to 100 vol. %, from 95 to 100 vol. % or from 98 to 100 vol. %. In some embodiments, the inert gas may be a noble gas. For example, the inert gas may be argon, helium, or combinations thereof. In some embodiments, the inert gas may be nitrogen gas.

The conveying medium and/or cleaning medium also preferably is substantially dry. For example, the conveying medium and/or cleaning medium may have a water concentration no greater than 80 wppm. In some embodiments, the water concentration of the conveying medium and/or the cleaning medium may be no greater than 80 wppm, no greater than 75 wppm, or no greater than 70 wppm. In some embodiments, the conveying medium and/or cleaning medium may have a dew point no greater than −40 °C. For example, the dew point temperature may be about −40 °C, about −45 °C, or about −50 °C. In some embodiments, the dew point temperature may be from about −80 °C to about −65 °C.

In some embodiments, the conveying medium and/or cleaning medium may have an oxygen concentration less than 1.000 vol. %, e.g., less than 0.500 vol. %, less than 0.250 vol. %, less than 0.100 vol. %, less than 0.050 vol %, or less than 0.010 vol. %. In terms of ranges, the conveying medium and/or cleaning medium may have an oxygen concentration from 0.001 to 1.000 vol. %, based on the total volume of the respective conveying medium and/or cleaning medium. For example, the oxygen concentration of the conveying medium and/or cleaning medium may be from 0.001 to 1.000 vol. %, from 0.005 to 1.000 vol. %, from 0.010 to 1.000 vol. %, from 0.050 to 1.000 vol. %, from 0.100 to 1.000 vol. %, from 0.500 to 1.00 vol. %, from 0.001 to 0.500 vol. %, from 0.001 to 0.100 vol. %, from 0.001 to 0.050 vol. %, from 0.001 to 0.010 vol. %, or from 0.001 to 0.005 vol. %. Additionally or alternatively, the oxygen content of the conveying medium and/or cleaning medium may be from about 0.002 vppm to about 0.007 vppm, e.g., about 0.002 vppm, about 0.003 vppm, about 0.004 vppm, about 0.005 vppm, about 0.006 vppm, about 0.007 vppm, or any value within a range between any two stated values.

As used herein, an “aqueous polymer dispersion” or “aqueous dispersion” may be a feed stock suitable for spray drying and preferably comprises one or more base polymers. Suitable base polymers for formation of the dispersion powders are those based on one or more ethylenically unsaturated monomers selected from the group comprising vinyl esters of carboxylic acids having 1 to 15 carbon atoms, methacrylic esters or acrylic esters of carboxylic acids with unbranched or branched alcohols having 1 to 15 carbon atoms, olefins or dienes, vinylaromatics or vinyl halides.

Preferred vinyl esters are vinyl acetate, vinyl propionate, vinyl butyrate, vinyl 2-ethylhexanoate, vinyl laurate, 1-methylvinyl acetate, vinyl pivalate and vinyl esters of alpha-branched monocarboxylic acids having 5 to 13 carbon atoms, for example VeoVa9 or VeoVa10 (trade names of Hexion). Particular preference is given to vinyl acetate.

Preferred methacrylic esters or acrylic esters are esters of unbranched or branched alcohols having 1 to 15 carbon atoms, such as methyl acrylate, methyl methacrylate, ethyl acrylate, ethyl methacrylate, propyl acrylate, propyl methacrylate, n-butyl acrylate, n-butyl methacrylate, 2-ethylhexyl acrylate, norbornyl acrylate. Particular preference is given to methyl acrylate, methyl methacrylate, n-butyl acrylate and 2-ethylhexyl acrylate.

Preferred olefins or dienes are ethylene, propylene and 1,3-butadiene. Preferred vinylaromatics are styrene and vinyltoluene. A preferred vinyl halide is vinyl chloride.

It is optionally also possible to copolymerize 0.05 to 50% by weight, preferably 1 to 10% by weight, based on the total weight of the base polymer, of auxiliary monomers. Examples of auxiliary monomers are ethylenically unsaturated mono-and dicarboxylic acids, ethylenically unsaturated carboxamides, ethylenically unsaturated sulfonic acids or salts thereof, precrosslinking comonomers such as polyethylenically unsaturated comonomers, postcrosslinking comonomers such as N-methylolacrylamide, epoxy-functional comonomers, silicon-functional comonomers.

Examples of suitable homo-and copolymers are vinyl acetate homopolymers, copolymers of vinyl acetate with ethylene, copolymers of vinyl acetate with ethylene and one or more further vinyl esters, copolymers of vinyl acetate with one or more further vinyl esters, copolymers of vinyl acetate with ethylene and acrylic esters, copolymers of vinyl acetate with ethylene and vinyl chloride, copolymers of vinyl chloride and ethylene and optionally one or more further vinyl esters, styrene-acrylic ester copolymers, styrene-1,3-butadiene copolymers.

Preference is given to vinyl acetate homopolymers; copolymers of vinyl acetate with 1 to 50% by weight of one or more further comonomers from the group of the vinyl esters having 1 to 12 carbon atoms in the carboxyl radical, such as vinyl propionate, vinyl laurate, vinyl esters of alpha-branched carboxylic acids having 5 to 13 carbon atoms, such as VeoVa9, VeoVa10, VeoVa11; copolymers of vinyl acetate with 1 to 40% by weight of ethylene; copolymers of vinyl acetate with 1 to 40% by weight of ethylene and 1 to 50% by weight of one or more further comonomers from the group of the vinyl esters having 1 to 12 carbon atoms in the carboxyl radical, such as vinyl propionate, vinyl laurate, vinyl esters of alpha-branched carboxylic acids having 5 to 13 carbon atoms, such as VeoVa9, VeoVa10, VeoVa11; copolymers of vinyl acetate, 1 to 40% by weight of ethylene and preferably 1 to 60% by weight of (meth)acrylic esters of unbranched or branched alcohols having 1 to 15 carbon atoms, especially methyl methacrylate, methyl acrylate, n-butyl acrylate or 2-ethylhexyl acrylate; copolymers with 30 to 75% by weight of vinyl acetate, 1 to 30% by weight of vinyl laurate or vinyl esters of an alpha-branched carboxylic acid having 5 to 13 carbon atoms, and 1 to 30% by weight of (meth)acrylic esters of unbranched or branched alcohols having 1 to 15 carbon atoms, especially methyl methacrylate, methyl acrylate, n-butyl acrylate or 2-ethylhexyl acrylate, which may also contain 1 to 40% by weight of ethylene; copolymers with one or more vinyl esters having 1 to 12 carbon atoms in the carboxyl radical, such as vinyl acetate, vinyl propionate, vinyl laurate, vinyl esters of alpha-branched carboxylic acids having 5 to 13 carbon atoms, such as VeoVa9, VeoVa10, VeoVa11, 1 to 40% by weight of ethylene and 1 to 60% by weight of vinyl chloride; where the polymers may each also contain the auxiliary monomers mentioned in the amounts mentioned, and the figures in % by weight add up to 100% by weight in each case.

Preference is also given to (meth)acrylic ester polymers, such as copolymers of n-butyl acrylate or 2-ethylhexyl acrylate or copolymers of methyl methacrylate with n-butyl acrylate and/or 2-ethylhexyl acrylate and optionally ethylene; styrene-acrylic ester copolymers with one or more monomers from the group of methyl acrylate, ethyl acrylate, propyl acrylate, n-butyl acrylate, 2-ethylhexyl acrylate; styrene-1,3-butadiene copolymers; where the polymers may also contain the auxiliary monomers mentioned in the amounts mentioned, and the figures in % by weight add up to 100% by weight in each case.

The monomer selection and the selection of the proportions by weight of the comonomers is generally made so as to result in a glass transition temperature Tg of −50 °C to +50 °C. The glass transition temperature Tg of the polymers can be determined in a known manner by means of differential scanning colorimetry (DSC). The Tg can also be calculated beforehand as an approximation by means of the Fox equation. According to Fox T. G., Bull. Am. Physics Soc. 1, 3, page 123 (1956): 1/Tg=x₁/Tg₁+x₂/Tg₂+ . . . +xₙ/Tgₙ, where xₙ is the mass fraction (% by wt./100) of the monomer n, and Tgₙ is the glass transition temperature in Kelvin of the homopolymer of the monomer n. Tg values for homopolymers are listed in Polymer Handbook, 2nd Edition, J. Wiley & Sons, New York (1975).

The base polymers are preferably prepared by an emulsion polymerization process. The polymerization temperature is preferably between 40 °C and 130 °C, more preferably between 60 °C and 100 °C. In the copolymerization of gaseous comonomers such as ethylene, 1,3-butadiene or vinyl chloride, it is also possible to work under pressure, generally between 5 bar and 100 bar. The polymerization is preferably initiated with the water-soluble initiators, thermal initiators, redox initiator, or combinations thereof, which are commonly used for emulsion polymerization.

The polymerization preferably is effected in the presence of protective colloids and/or emulsifiers. Suitable protective colloids for the polymerization are one or more of polyvinyl alcohols; polyvinyl acetals; polyvinylpyrrolidones; polysaccharides in water-soluble form, such as starches (amylose and amylopectin), celluloses and the carboxymethyl, methyl, hydroxyethyl, hydroxypropyl derivatives thereof, dextrins and cyclodextrins; proteins such as casein or caseinate, soya protein, gelatin; lignosulfonates; synthetic polymers such as poly(meth)acrylic acid, copolymers of (meth)acrylates with carboxy-functional comonomer units, poly(meth)acrylamide, polyvinylsulfonic acids and the water-soluble copolymers thereof; melamine-formaldehydesulfonates, naphthalene-formaldehydesulfonates, styrene-maleic acid and vinyl ether-maleic acid copolymers. Preference is given to using partly hydrolyzed or fully hydrolyzed polyvinyl alcohols having a hydrolysis level of 80 to 100 mol %, especially partly hydrolyzed polyvinyl alcohols having a hydrolysis level of 80 to 95 mol % and a Hoeppler viscosity of 1 to 30 mPa*s (Hoeppler method at 20 °C, DIN 53015) in 4% aqueous solution.

If polymerization is effected in the presence of emulsifiers, the amount thereof is 1 to 5% by weight, based on the amount of monomers. Suitable emulsifiers are anionic, cationic or nonionic emulsifiers, for example anionic surfactants such as alkyl sulfates having a chain length of 8 to 18 carbon atoms, alkyl or alkylaryl ether sulfates having 8 to 18 carbon atoms in the hydrophobic radical and up to 40 ethylene oxide or propylene oxide units, alkyl-or alkylarylsulfonates having 8 to 18 carbon atoms, esters and monoesters of sulfosuccinic acid with monohydric alcohols or alkylphenols, or nonionic surfactants such as alkyl polyglycol ethers or alkylaryl polyglycol ethers having 8 to 40 ethylene oxide units.

On completion of the polymerization, residual monomers can be removed by postpolymerization employing known methods, generally by redox catalyst-initiated postpolymerization. Volatile residual monomers can also be removed by means of distillation, preferably under reduced pressure, and optionally while passing inert entraining gases through or over, such as air, nitrogen or water vapor.

The aqueous polymer dispersions thus obtainable optionally have a solids content of 30 to 75% by weight, e.g., 50 to 60% by weight. Additional aqueous polymer dispersions suitable for use in the spray drying processes described herein are described, for example, in U.S. Pat. Nos. 8,844,783B2; 9,334,388B2; and 9,353,005B2, the entireties of which are incorporated herein by reference.

### Spray Drying Device

As mentioned above, spray drying is a process in which a liquid feed, usually an aqueous polymer dispersion, as described above, is converted into a powder. Advantageously, spray drying may produce fine particles of controlled and uniform size. Due to the ability to produce highly controlled particles, spray drying is utilized in a variety of industries which require consistent particle formation including, but not limited to, food production, pharmaceuticals, chemical manufacturing, and materials sciences.

Therefore, in one aspect, the disclosure relates to a rotary spray dryer comprising a vessel defining a drying chamber, wherein the vessel comprises a rotary atomizer configured to atomize an aqueous polymer dispersion to form an atomized dispersion; a drying medium inlet in fluid communication with the drying chamber configured to dry the atomized dispersion with a drying medium and form solid particles; and an anti-blocking agent inlet configured to deliver an anti-blocking agent to the drying chamber with a conveying medium comprising an inert gas in an amount greater than 90 vol. %, based on the total volume of the conveying medium.

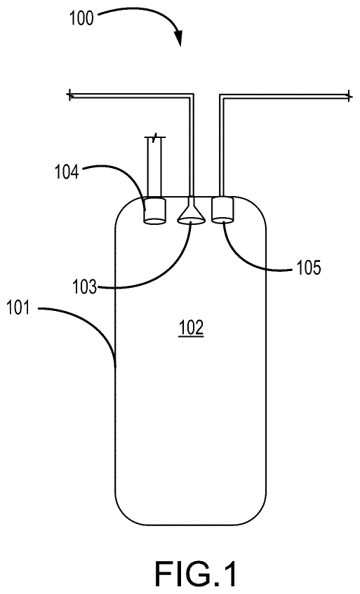

FIG. 1 shows an exemplary rotary spray drying device 100 comprising a vessel 101 defining a drying chamber 102. The vessel may comprise a rotary atomizer 103 configured to atomize an aqueous polymer dispersion to form an atomized dispersion. In general, a rotary atomizer may use a high-speed rotating disk, cup, or wheel to discharge an aqueous polymer dispersion, thereby creating an atomized aqueous polymer dispersion, which quickly dries forming finely dispersed particles. For example, a rotary atomizer may be a high-speed spinning disk and/or wheel that uses centrifugal force to break an aqueous polymer dispersion into fine droplets. In some embodiments, the atomizer may comprise a perforated disk, a nozzle disk, a plate disk, or comprise an atomizer cap or be a multi-row perforated disk (perforated cylinder). Depending on application and composition of the aqueous dispersion, other atomizing techniques or devices may be used to atomize the aqueous polymer dispersion. For example, a pressure nozzle, a two-fluid nozzle, an electrostatic atomizer, or an air atomization may be used.

A drying medium inlet 104 in fluid communication with the drying chamber may be configured to dry the atomized polymer dispersion with a drying medium and form particles. The drying medium inlet should be oriented such that the drying medium contacts the atomized dispersion, ideally as it is formed. As used herein, contacting may be used to refer to the instant the atomized dispersion come into direct contact with the drying medium in the drying chamber. By way of example, contacting may result in rapid evaporation of a liquid from at atomized dispersion, wherein the liquid may be water (e.g., for aqueous polymer dispersions) and/or volatile components. In some embodiments, volatile components may include low molecular weight compounds such as ethanol, acetaldehyde, diacetyl, limonene, ethyl acetate, as well as alcohols, aldehydes, ketones, and esters generally.

In some embodiments, the drying medium may be heated by an external source to accelerate the drying process. For example, the drying gas may be heated by passing through a heat exchanger wherein the heat may be supplied by, without limitation, steam, hot oil, electrical heating, and/or gas combustion. In some embodiments, the drying medium may be heated to a temperature of about 100 °C to about 200 °C, as measured at the drying medium inlet. In some embodiments, the drying medium may be heated to a temperature of from about 60 °C to about 95 °C.

The composition of the drying medium may vary widely. In some aspects, the drying medium may comprise or consist essentially of air. Although less practical owing to the volume of drying medium typically required, in other aspects, the drying medium may comprise an inert gas, optionally in an amount greater than 80 vol. %, e.g., greater than 90 vol. %, or greater than 95 vol. %, or greater than 99 vol. %. Since its purpose is to dry the feed, e.g., the aqueous polymer dispersion, the drying medium ideally is substantially dry as it is fed to the drying chamber, optionally comprising water vapor in an amount less than 4.5 vol. %, e.g., less than 3.5 vol. %, less than 2.5 vol. %, or less than 1.5 vol. %. In some embodiments, the drying medium may have a water vapor in an amount less than 300 vppm, e.g., less than 270 vppm, or less than 250 vppm.

In some aspects, an anti-blocking agent may be introduced into the drying chamber to control and/or enhance product particle properties. An anti-blocking agent inlet 105 may be in fluid communication with a conveying medium source and an anti-blocking agent source. In some embodiments, the conveying medium source may be a pressurized vessel, e.g., a gas cylinder. For example, the conveying medium source may be pressurized to about 2,000 to about 3,000 pounds per square inch gauge (psig), i.e., about 1.4×10⁴ kPa to about 2.1×10⁴ kPa. The conveying medium may be configured to exert positive pressure on the anti-blocking agent source thereby conveying the anti-blocking agent. In some embodiments, there may be an in-line regulator which may control the conveying medium pressure before contacting the anti-blocking agent. For example, the conveying medium pressure may be about 2 to about 10 psig, i.e., about 14 kPa to about 69 kPa. Additionally or alternatively, there may be an in-line regulator that controls the rate of the anti-blocking agent and the conveying medium during conveyance. For example, the conveyance pressure may be about 2 to about 10 psig, i.e., about 14 kPa to about 69 kPa. The exemplary pressures described herein may be adjusted depending on several factors, including by not limited to, composition of the aqueous dispersion, desired product particle size, temperature of the drying medium, volume of the drying chamber, and/or type of atomization.

In some embodiments, the rotary atomizer, drying medium inlet, and/or the anti-blocking agent inlet may be at a first end of the vessel, and a product outlet may be at a second end of the vessel. For example, the vessel may be a cylindrical tower, wherein the aqueous dispersion is atomized and contacted with the drying medium and/or anti-blocking agent at the first end of the vessel and moved by positive pressure and/or gravity to the product outlet at the second end.

In some embodiments, the disclosed rotary spray drying device may comprise a vessel defining a drying chamber; a feed inlet conduit oriented at a first end of the vessel and in fluid communication with a liquid feed source and a drying gas source; a rotary atomizer oriented at an end of the feed inlet conduit within the vessel and configured to atomize a first feed from the liquid feed source with a drying gas from the drying gas source; an anti-blocking agent delivery mechanism configured to deliver an anti-blocking agent to the drying chamber with a conveying medium, wherein the atomized first feed and anti-blocking agent form particles in the drying chamber; and a product outlet configured to convey the particles to a particle collection device.

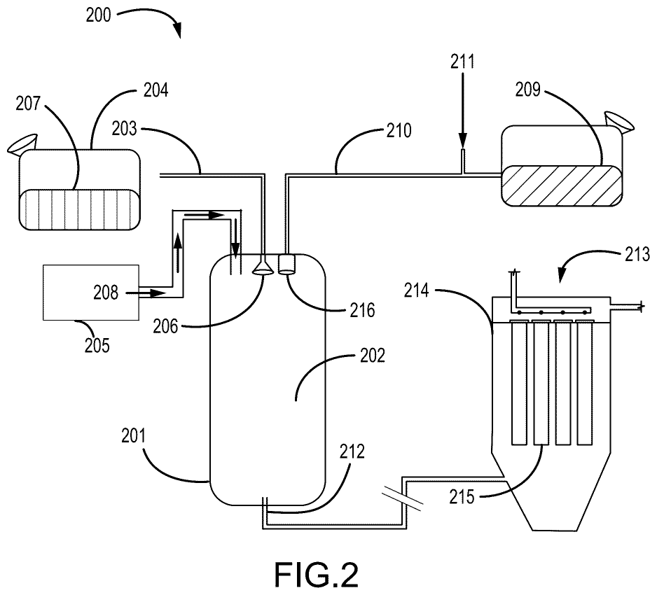

FIG. 2 shows an exemplary rotary spray dryer system 200 comprising a vessel 201 defining a drying chamber 202. A feed inlet conduit 203 may be oriented at a first end of the vessel and in fluid communication with a liquid feed source 204. A rotary atomizer 206 may be oriented at an end of feed inlet conduit 203 within the vessel and configured to atomize a first feed 207 from the liquid feed source with a drying gas 208 delivered into the drying chamber via drying gas source 205.

Depending on the application, additional additives may be incorporated into the spray drying process. In some embodiments, an anti-blocking agent may be added during the spray drying process to enhance efficiency and properties of the resulting product particles. Optionally, anti-blocking agent may be delivered, for example, into an annular gap formed by a concentric space around the rotary atomizer. Delivery in this location may result in processing challenges, e.g., reduced dispersion within the feed stream. Additionally or alternatively, however, anti-blocking agent may be delivered into a spray drying device outside of a concentric space around the rotary atomizer via one or more separate anti-blocking agent conduits, as shown in FIG. 2. For example, the anti-blocking agent may be delivered via one or more separate conduits to a space adjacent the rotary atomizer. In some embodiments, the anti-blocking agent delivery mechanism 216 may be configured to deliver anti-blocking agent 209 into a region adjacent the rotary atomizer 206 through a conduit 210. Optionally, in some embodiments, the anti-blocking may be delivered through a plurality of separate conduits.

Suitable anti-blocking agents are known to those skilled in the art and may include, for example, one or more of the following: aluminum silicates such as kaolin, bentonite, fumed silica or precipitated silica, which may optionally be hydrophobized, silicates having particle sizes preferably in the range of 10 nm to 100 μm, talc, clays, gypsum, kaolines, metakaolin, calcined kaolin, calcium sulfate, carbonates such as calcium carbonate, magnesium carbonate, Ca/Mg carbonate, dolomite, barium sulfate, polyvinyl alcohols, polyvinyl acetals, polyvinylpyrrolidones, polysaccharides in water-soluble form such as starches (amylose and amylopectin), celluloses and the carboxymethyl, methyl, hydroxyethyl, hydroxypropyl derivatives thereof, dextrin's and cyclodextrins, proteins such as casein or caseinate, soy protein, gelatin, lignosulfonates, synthetic polymers such as poly(meth)acrylic acid, copolymers of (meth)acrylates with carboxy-functional comonomer units, poly(meth)acrylamide, polyvinyl sulfonic acids and the water-soluble copolymers thereof, melamine-formaldehyde sulfonates, naphthalene-formaldehyde sulfonates, styrene-maleic acid copolymers and vinyl ether-maleic acid copolymers, water insoluble film forming polyaddition and polycondensation polymers such as polyurethanes, polyesters, polyether's, polyamides, melamine-formaldehyde resins, naphthalene-formaldehyde resins, phenol-formaldehyde resins, optionally also in the form of their oligomeric pre-products, or mixtures thereof. In some embodiments, the anti-blocking agent may be a mixture of calcium (Ca) and magnesium (Mg) carbonate and silica. In some embodiments, the anti-blocking agent may be a mixture of Kaolin and calcium carbonate. In some embodiments, the anti-blocking agent may be a mixture of Kaolin and dolomite. The anti-blocking agents are used generally in an amount of 0.1 to 30% by weight, preferably 2 to 30% by weight, more preferably 7 to 30% by weight, based in each case on the total weight of the polymeric constituents of the polymer dispersion to be atomized. In some embodiments, a mixture of anti-blocking agents may be used wherein the anti-blocking agents may be present individually in an amount from 0.05 to 29.95% by weight, e.g., from 0.1 to 25% by weight, from 1 to 20% by weight, from 6 to 15% by weight, or from 10 to 20% by weight, or in an amount equal to any of the aforementioned endpoints of such ranges, based on the total weight of the polymeric constituents of the polymer dispersion to be atomized. For example, in a specific example, the antiblocking agent may comprise a mixture of kaolin and calcium carbonate, e.g., comprising 6% by weight kaolin and 10% of weight calcium carbonate. In some embodiments, the anti-blocking agent may comprise at least one of functional additives and/or finished product rework. For example, the functional additives may comprise maltodextrin, gum arabic, formic acid, dextran, cellulose, cellulose derivatives, agar, carrageenan, gluten, caseins, albumins, peptides, and/or wax. In other aspects, the antiblocking agent may comprise an encapsulated silane powder such as, for example, a SEAL200-type silane powder; a vinyl acetate/ethylene copolymer redispersible powder, such as, for example, HD2000, HD2040, or FLOWKIT53, or a PCE (polycarboxylate ether) powder, such as, for example, FLOW 821.

As described herein, the use of compressed air to convey anti-blocking agents in conventional methods may result in agglomeration. Agglomeration may be undesirably enhanced due to water vapor contained in the compressed air. Without being bound by theory, water may facilitate the formation of liquid bridges between individual particles, i.e., when water contacts the surface of two adjoining particles capillary forces may bring them together, a union which multiplies as more particles are contacted thereby forming agglomerates. Therefore, the presence of water in an anti-blocking conveyance stream may result in the agglomeration of the anti-blocking agent itself, the atomized first feed, and/or the anti-blocking agent and the atomized first feed. This problem may be exacerbated in manufacturing facilities located in humid environments. To address this, in some embodiments the disclosure relates to the use of a conveying medium 211 to deliver anti-blocking agent 209 into the drying chamber.

As discussed above, the conveying medium ideally is substantially dry. For example, the conveying medium optionally comprises water in an amount less than 500 wppm, less than 250 wppm, less than 100 wppm, less than 80 wppm, or less than 75 wppm. In terms of ranges, the conveying medium may have a water concentration from 75 to 500 wppm, from 75 to 250 wppm, from 75 to 100 wppm, from 75 to 100 wppm, or from 75 to 80 wppm.

Dew point is the temperature at which a gas becomes saturated with water vapor. Therefore, dew point can be related to water concentration. In some embodiments, the conveying medium may be characterized in terms of this property. For example, in some aspects, the conveying medium may have a dew point temperature (DP) no greater than −40 °C. For example, the dew point temperature may be about −40 °C, about −45 °C, or about −50 °C. In some embodiments, the dew point temperature (DP) may be from −80 °C to −65 °C

During the spray drying process, including conveyance of materials to and from the system, friction caused by particles and dust rubbing against one another and/or components of the device may generate a charge buildup, i.e., static electricity. The developed charge may ultimately be released, i.e., static discharge, which may result in ignition of the particles and/or dust. Ignition in the presence of oxygen may lead to an explosion. Therefore, in some embodiments, the present disclosure relates to a spray drying device wherein a conveying medium may be used to convey the anti-blocking agent. In some embodiments, the conveying medium may be a noble gas. For example, the noble gas may be helium and/or argon. In some embodiments, the conveying medium may be nitrogen.

The use of a conveying medium may not only reduce the likelihood of ignition, it may also decrease flame propagation if ignition does occur. In some embodiments, the conveying medium may have an oxygen concentration from 0.001 to 1.000 vol. %, based on a total volume of the conveying medium. For example, the oxygen concentration of the conveying medium may be about 0.001 to 1.000 vol. %, about 0.010 to 1.000 vol. %, about 0.100 to 1.000 vol. %. Additionally or alternatively, the oxygen content of the conveying medium may be from about 0.002 vppm to about 0.007 vppm, e.g., about 0.002 vppm, about 0.003 vppm, about 0.004 vppm, about 0.005 vppm, about 0.006 vppm, about 0.007 vppm, or any value within a range between any two stated values.

Once particles have formed in the spray drying device 200, a product outlet 212 may be configured to convey the particles to a particle collection device 213. In some embodiments, the particle collection device may comprise a filter housing 214 and a bag filter 215. In some embodiments, filter housing may comprise a plurality of bag filters.

### Process for Producing a Dispersion Powder

In another aspect, the disclosure relates to a process for producing a dispersion powder comprising atomizing a quantity of an aqueous polymer dispersion and adding an anti-blocking agent in a rotary spray dryer, wherein the anti-blocking agent is conveyed by a conveying medium comprising an inert gas in an amount greater than 90 vol. %, based on the total volume of the conveying medium.

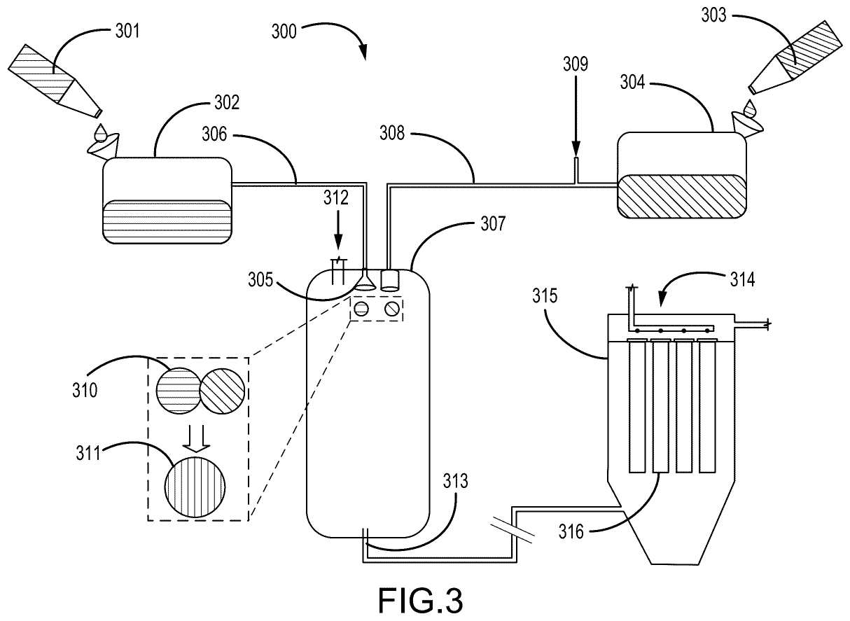

FIG. 3 shows an exemplary process for producing a dispersion powder, comprising: providing a first feed to a liquid feed source; providing an anti-blocking agent to an anti-blocking agent hopper; conveying the anti-blocking agent from the hopper to a drying chamber with a conveying medium into a region adjacent to a rotary atomizer spray head; conveying the first feed to the rotary atomizer spray head through a feed inlet conduit and atomizing the first feed into the drying chamber; contacting the atomized first feed with the anti-blocking agent and a drying gas, thereby forming a particles in the drying chamber; and conveying the particles through a product outlet to a particle collection device.

Beneficially, the process described herein may provide for the more efficient preparation of particles, safer operating conditions, and increased system performance. In some embodiments, the process for producing a dispersion powder may utilize rotary spray drying systems 100 or 200. In some embodiments, the process may utilize a rotary spray drying system that includes other components and/or omits some features described in relation to rotary spray drying systems 100 or 200. As illustrated in FIG. 3, in some embodiments the process for producing a dispersion powder 300 may comprise providing a first feed 301 to a liquid feed source 302 and providing an anti-blocking agent 303 to an anti-blocking agent hopper 304. The composition of the anti-blocking agent may be as described above.

As illustrated in FIG. 3, the first feed may be conveyed to a rotary atomizer spray head 305 through a feed inlet conduit 306, and the anti-blocking agent may be conveyed from the hopper to a drying chamber 307, through conduit 308, into a region adjacent to a rotary atomizer spray head. In some embodiments, the anti-blocking agent is delivered through a plurality of separate conduits. In some embodiments, the anti-blocking agent may be delivered into the concentric space, e.g., annular space, around the rotary atomizer.

In an aspect of the present disclosure, the conveyance of the of the anti-blocking agent may be with conveying medium 309. In some embodiments, the conveying medium may comprise an inert gas in an amount greater than 90 vol. %, based on the total volume of the conveying medium, as described above.

As shown in FIG. 3 upon conveyance of the anti-blocking agent into the region adjacent to the rotary atomizer, anti-blocking agent may contact the atomize first feed 310. The atomized first feed and anti-blocking agent may then be contacted with drying gas 312. Contacting may produce particles 311, comprising anti-blocking agent and atomized first feed, in the drying chamber. As disclosed herein, delivery of the anti-blocking agent into a region adjacent to the spray head may provide beneficial contacting of the anti-blocking agent with the atomized first feed, resulting in increased particle formation. Particle 311 may then be conveyed through a product outlet 313, to a particle collection device 314. In some embodiments, the particle collection device may comprise a filter housing 315 and a bag filter 316. In some embodiments, the particle collection device may comprise a plurality of bag filters.

### Method for Cleaning a Particle Collection Device

As has been described herein, conventional spray drying devices and methods may produce dust. Accumulation of dust within a rotary spray drying system can lead to an increased risk of explosion. To prevent such problems, a particle collection device may be employed to capture dust from the production stream, ultimately leading to dust accumulation in the particle collection device. Dust accumulation in the particle collection device may undesirably reduce airflow through the spray drying device, again resulting in the aforementioned safety and efficiency concerns. Therefore, mitigation of dust accumulation within the particle collection device must be addressed. One method to mitigate dust accumulation is to employ compressed air to dislodging dust particles from the bag filters within, which leads to the regeneration of dispersed dust. The rapid production of suspended dust in combination with the high oxygen concentration of compressed air may increase the likelihood of an explosion. The inventors have discovered a stream of a cleaning medium to a particle collection device effectively mitigates dust accumulation while also ameliorating safety concerns related to the use of compressed air.

In a further aspect, the disclosure relates to a method for cleaning a particle collection device comprising, providing stream of a cleaning medium to a particle collection device, wherein the cleaning medium comprises at least 90 vol. % of an inert gas, based on a total volume of the cleaning medium. Additional optional compositions for the cleaning medium are discussed above.

Dust and product particles may accumulate in a particle collection device during the spray drying process. By way of example, a particle collection device may comprise a plurality of bag filters which separate the production stream from the conveying medium. This separation process may result in the accumulation of product particles and dust on the surface of the bag filters, leading to pressure drop across the system and therefore reduced efficiency. Additionally, the mitigation of accumulation from the surface of bag filters may result in regeneration of suspended particles, thereby increasing the risk of ignition and explosion. This risk may be increased when utility compressed air is used. Beneficially, the present inventors have discovered the use of a cleaning medium comprising an inert gas abrogates the aforementioned safety concerns allowing for effective cleaning and increased spray drying efficiency.

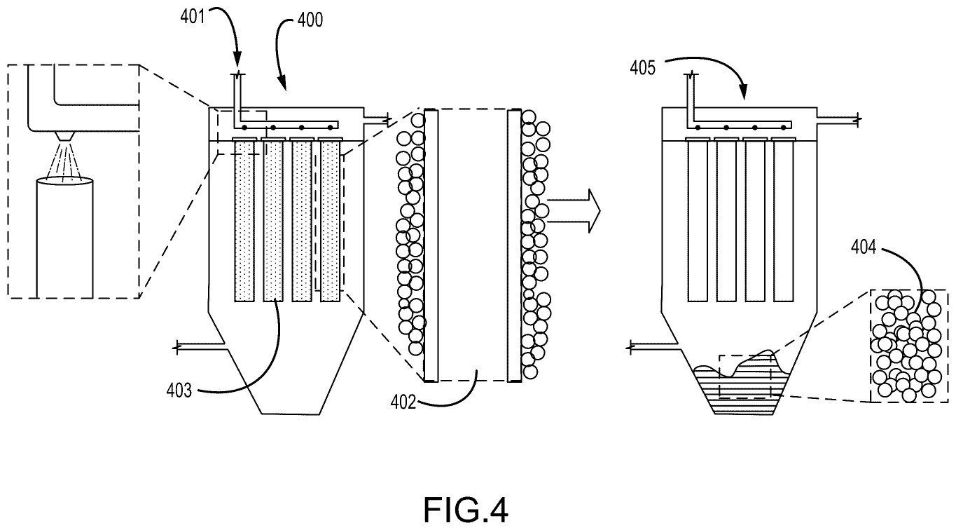

FIG. 4 shows an exemplary method for cleaning a particle collection device according to the present disclosure. Particle collection device 400 may comprise a plurality of bag filters 403. A stream of cleaning medium 401 may be provided to the particle collection device 400, and may be directed into each individual bag filter, thereby causing the product particles and/or dust 402 to dislodge from the surface of bag filter 403. Once dislodged, product particles and/or dust 404 may then be safely removed from the now clean particle collection device 405. In some embodiments, a stream of cleaning medium may be provided to each filter housing. In some embodiments, the stream of cleaning medium may be at a rate of about 10 L/second to about 100 L/second. In some embodiments, the stream of cleaning medium may be released for about 0.5 seconds to about 3.0 seconds. In some embodiments, the stream of cleaning medium may be at a rate of about 65 L/second for about 0.5 seconds.

### Embodiments

As used below, any reference to a series of Embodiments is to be understood as a reference to each of those examples disjunctively (e.g., “Embodiments 1-4” is to be understood as “Embodiments 1, 2, 3, or 4”).
Embodiment 1: A rotary spray dryer system, comprising: a vessel defining a drying chamber, wherein the vessel comprises a rotary atomizer configured to atomize an aqueous polymer dispersion to form an atomized dispersion; a drying medium inlet in fluid communication with the drying chamber, configured to dry the atomized dispersion with a drying medium and form particles; and an anti-blocking agent inlet configured to deliver an anti-blocking agent to the drying chamber with a conveying medium comprising an inert gas in an amount greater than 90 vol. %, based on the total volume of the conveying medium.
Embodiment 2: The rotary spray dryer system of Embodiment 1, wherein the anti-blocking agent inlet is oriented adjacent to the rotary atomizer.
Embodiment 3: The rotary spray drying system of Embodiment 1 or 2, wherein the anti-blocking agent inlet comprises a plurality of separate conduits.
Embodiment 4: The rotary spray drying system of Embodiment 1, wherein the anti-blocking agent inlet is oriented into an annular gap formed by a concentric space around the rotary atomizer.
Embodiment 5: The rotary spray drying system of any of the preceding Embodiments, wherein the anti-blocking agent comprises at least one of functional additives, filler, finished product rework, or combinations thereof.
Embodiment 6: The rotary spray drying system of any of the preceding Embodiments, wherein the inert gas is a noble gas.
Embodiment 7: The rotary spray drying system of any of the preceding Embodiments, wherein inert gas is nitrogen gas.
Embodiment 8: The rotary spray drying system of any of the preceding Embodiments, wherein the inert gas has a water concentration no greater than 80 wppm.
Embodiment 9: The rotary spray drying system of any of the preceding Embodiments, wherein the inert gas has a dew point temperature no greater than −40 °C.
Embodiment 10: The rotary spray drying system of any of the preceding Embodiments, wherein the inert gas has an oxygen concentration less than 1.000 vol %, based on the total volume of the conveying medium.
Embodiment 11: The rotary spray drying system of any of the preceding Embodiments, further comprising a particle collection device comprising a filter housing and a bag filter.
Embodiment 12: A process for producing a dispersion powder comprising the steps: of atomizing a quantity of an aqueous polymer dispersion and adding an anti-blocking agent in a rotary spray dryer, wherein the anti-blocking agent is conveyed by a conveying medium comprising an inert gas in an amount greater than 90 vol. %, based on the total volume of the conveying medium.
Embodiment 13: The process of producing a dispersion powder of Embodiment 12, wherein the inert gas is a noble gas.
Embodiment 14: The process of producing a dispersion powder of Embodiment 12, wherein the inert gas is nitrogen gas.
Embodiment 15: The process of producing a dispersion powder of any of Embodiments 12-14, wherein anti-blocking agent comprises at least one of functional additives, filler, finished product rework, or combinations thereof.
Embodiment 16: The process of producing a dispersion powder of any of Embodiments 12-15, wherein the inert gas has a water concentration no greater than 80 wppm.
Embodiment 17: The process of producing a dispersion powder of any of Embodiments 12-16, wherein the inert gas has a dew point temperature no greater than −40 °C.
Embodiment 18: The process of producing a dispersion powder of any of Embodiments 12-17, wherein the inert gas has an oxygen concentration less than 1.000 vol %, based on the total volume of the conveying medium.
Embodiment 19: A method for cleaning a particle collection device, comprising: providing stream of a cleaning medium to the particle collection device, wherein the cleaning medium comprises at least 90 vol. % of an inert gas, based on a total volume of the cleaning medium.
Embodiment 20: The method for cleaning a particle collection device of Embodiment 19, wherein the inert gas is a noble gas.
Embodiment 21: The method for cleaning a particle collection device of Embodiment 19, wherein the inert gas is nitrogen gas.
Embodiment 22: The method for cleaning a particle collection device of any of Embodiments 19-21, wherein the stream of cleaning medium is at a rate of about 10 L/second to about 100 L/second for about 0.1 to about 3.0 seconds.
Embodiment 23: The method for cleaning a particle collection device of any of Embodiments 19-22, wherein the inert gas has a water concentration no greater than 80 wppm.
Embodiment 24: The method for cleaning a particle collection device of any of Embodiments 19-23, wherein the inert gas has a dew point temperature no greater than −40 °C.
Embodiment 25: The method for cleaning a particle collection device of any of Embodiments 19-24, wherein the inert gas has an oxygen concentration less than about 1.000 vol. %, based on a total volume of the cleaning medium

All references throughout this application, for example patent documents, including issued or granted patents or equivalents and patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising,” particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A rotary spray dryer system, comprising:
a vessel defining a drying chamber, wherein the vessel comprises a rotary atomizer configured to atomize an aqueous polymer dispersion to form an atomized dispersion;
a drying medium inlet in fluid communication with the drying chamber configured to dry the atomized dispersion with a drying medium and form particles; and
an anti-blocking agent inlet configured to deliver an anti-blocking agent to the drying chamber with a conveying medium comprising an inert gas in an amount greater than 90 vol. %, based on the total volume of the conveying medium.

2. The rotary spray dryer system of claim 1, wherein the anti-blocking agent inlet comprises one or more conduits oriented adjacent to the rotary atomizer.

3. The rotary spray drying system of claim 1, wherein the anti-blocking agent inlet is oriented into an annular gap formed by a concentric space around the rotary atomizer.

4. The rotary spray drying system of claim 1, wherein the anti-blocking agent comprises at least one of functional additives, filler, finished product rework, or combinations thereof.

5. The rotary spray drying system of claim 1, wherein the inert gas is a noble gas or nitrogen.

6. The rotary spray drying system of claim 1, wherein the conveying medium has a water concentration no greater than 80 wppm.

7. The rotary spray drying system of claim 1, wherein the conveying medium has a dew point temperature no greater than −40 °C.

8. The rotary spray drying system of claim 1, wherein the conveying medium has an oxygen concentration less than 1.000 vol %, based on the total volume of the conveying medium.

9. A process for producing a dispersion powder comprising atomizing a quantity of an aqueous polymer dispersion and adding an anti-blocking agent in a rotary spray dryer, wherein the anti-blocking agent is conveyed by a conveying medium comprising an inert gas in an amount greater than 90 vol. %, based on the total volume of the conveying medium.

10. The process of producing a dispersion powder of claim 9, wherein anti-blocking agent comprises at least one of functional additives, filler, finished product rework, or combinations thereof.

11. The process of producing a dispersion powder of claim 9, wherein the inert gas is a noble gas or nitrogen.

12. The process of producing a dispersion powder of claim 9, wherein the conveying medium has a water concentration no greater than 80 wppm.

13. The process of producing a dispersion powder of claim 9, wherein the conveying medium has a dew point temperature no greater than −40 °C.

14. The process of producing a dispersion powder of claim 9, wherein the conveying medium has an oxygen concentration less than 1.000 vol %, based on the total volume of the conveying medium.

15. A method for cleaning a particle collection device, comprising:
providing a burst of a cleaning medium to a particle collection device, wherein the cleaning medium comprises an inert gas in an amount greater than 90 vol. %, based on a total volume of the cleaning medium.

16. The method for cleaning a particle collection device of claim 15, wherein the burst flows at a rate of about 10 L/second to about 100 L/second for about 0.1 seconds to about 3.0 seconds.

17. The method for cleaning a particle collection device of claim 15, wherein the inert gas is a noble gas or nitrogen.

18. The method for cleaning a particle collection device of claim 15, wherein the cleaning medium has a water concentration no greater than 80 wppm.

19. The method for cleaning a particle collection device of claim 15, wherein the cleaning medium has a dew point temperature no greater than −40 °C.

20. The method for cleaning a particle collection device of claim 15, wherein the cleaning medium has an oxygen concentration less than about 1.000 vol. %, based on a total volume of the cleaning medium.
